(19) United States
(12) Patent Application Publication
(10) Pub. No.: US 2023/0364156 A1
(43) Pub. Date: Nov. 16, 2023

(54) TREATING OF NEUROLOGICAL DISORDERS USING EXTRACELLULAR VESICLES RELEASED BY HUMAN INDUCED PLURIPOTENT STEM CELL DERIVED NEURAL STEM CELLS

(71) Applicant: THE TEXAS A&M UNIVERSITY SYSTEM, College Station, TX (US)

(72) Inventors: Ashok K. SHETTY, College Station, TX (US); Raghavendra UPADHYA, College Station, TX (US); Leelavathi N. MADHU, College Station, TX (US); Sahithi ATTALURI, College Station, TX (US); Maheedhar KODALI, College Station, TX (US)

(21) Appl. No.: 18/318,305

(22) Filed: May 16, 2023

Related U.S. Application Data
(60) Provisional application No. 63/342,276, filed on May 16, 2022.

(51) Int. Cl. A61K 35/545; A61K 9/00; A61K 45/06; A61P 25/28

(52) U.S. Cl. CPC A61K 35/545; A61K 9/0043; A61K 45/06; A61P 25/28

(57) ABSTRACT

A method for treating a neurological disorder, comprising administering a therapeutically effective amount of extracellular vesicles (EVs) derived from human induced pluripotent stem cell derived neural stem cells (hiPSC-NSCs) to a subject in need thereof. The neurological disorders may, for example, be Alzheimer's disease, Parkinson's disease, Huntington's disease, amyotrophic later sclerosis (ALS), multiple sclerosis, dementia, traumatic brain injury or stroke. A composition comprising EVs derived from hiPSC-NSCs. A kit comprising EVs derived from hiPSC-NSCs.

### CROSS-REFERENCE TO RELATED APPLICATIONS

This application claims the benefit under 35 U.S.C. § 119(e) of U.S. Provisional Application Ser. No. 63/342,276, filed on May 16, 2022, the entire disclosure of which is incorporated herein by reference.

### BACKGROUND OF THE INVENTION

Neurological disorders are diseases that cause progressive damage to the brain and nervous system. These diseases are characterized by the loss of nerve cells, which can lead to a variety of symptoms, including memory loss, difficulty thinking, impaired movement, and changes in mood and behavior. Examples of neurological disorders include Alzheimer's disease, Parkinson's disease, Huntington's disease, amyotrophic later sclerosis, multiple sclerosis, dementia, traumatic brain injury or stroke.

Alzheimer's disease (AD) is a progressive neurodegenerative disorder characterized by loss of memory and cognitive function. It is the leading cause of dementia in older adults and is the sixth leading cause of death in the United States. More than 6 million people in the United States have Alzheimer's disease.

Parkinson's disease (PD) affects movement. It is characterized by tremors, stiffness, and impaired balance. About 1 million people in the United States have Parkinson's disease.

Huntington's disease is an inherited neurodegenerative disorder that causes progressive damage to nerves cells in the brain. Symptoms include involuntary movements and decline in cognitive, emotional, and motor functions. About 30,000 people in the United States have Huntington's disease.

Amyotrophic lateral sclerosis (ALS) affects nerves cells in the brain and spinal cord, leading to muscle weakness and wasting, and eventually paralysis. Approximately 18,000 people in the United States have ALS.

Multiple sclerosis (MS) is a chronic autoimmune disease that affects the central nervous system (CNS). In MS, the immune system attacks the myelin sheath that surrounds and protects nerve fibers. The damage can lead to a variety of symptoms such as fatigue, muscle weakness, and problems with balance and coordination. Approximately 1 million people in the United States have MS.

Dementia is a general term for loss of cognitive function, such as memory, thinking, and judgment. Dementia may be caused by a variety of conditions, including Alzheimer's disease, Parkinson's disease, and Huntington's disease. About 5.8 people in the United States gave dementia.

Traumatic brain injury (TBI) is damage or disruption to the brain caused by an external force or trauma. Approximately 5.3 million people in the United States have a TBI-related disability.

A stroke is a medical condition that occurs when the blood supply to a part of the brain is disrupted. The interruption of blood supply may lead to damage or death of brain cells. Every year, more than 795,000 people in the U.S. have a stroke.

There are currently no cures for any of the above disorders, though there are available treatments which can help manage the symptoms, slow the progression of the disease, and improve the quality of life for the patient.

Human induced pluripotent stem cell (hiPSC)-derived neural stem cells (NSCs) are stem cells generated by reprogramming adult cells into a pluripotent state, enabling them to differentiate into various cell types, including neurons, astrocytes, and oligodendrocytes. Advantages of hiPSC-derived neural stem cells is that they can be grown in large quantities and that they can be generated from a patient's own cells, thus allowing for personalized therapy and reducing risk of immunogenicity.

Grafting of hiPSCs-derived NSCs has shown promise for brain repair after injury or disease, but safety issues, which include immunogenic risks, and the possible genetic instability leading to incomplete differentiation or teratoma formation, have hindered their clinical application.

Extracellular vesicles (EVs) are small membrane-bound particles released by cells, including NSCs. EVs contain a variety of proteins, lipids, nucleic acids, and other molecules and are typically involved in the transfer of such molecules between cells. The use of EVs in therapy has advantages over the use of cells in that they are much smaller and thus easier to deliver and can easily cross the blood-brain barrier. In addition, they are less likely to be rejected by the immune system. EVs can also be produced in even larger quantities than NSCs.

The present invention relates to the use of EV is released by hiPSCs-derived NSCs in the treatment of neurological disorders such as Alzheimer's disease, Parkinson's disease, Huntington's disease, amyotrophic later sclerosis, multiple sclerosis, dementia, traumatic brain injury, and stroke.

### SUMMARY OF THE INVENTION

The present invention relates in part to a method for treating a neurological disorder, comprising administering a therapeutically effective amount of extracellular vesicles (EVs) derived from human induced pluripotent stem cell derived neural stem cells (hiPSC-NSCs) to a subject in need thereof.

In certain embodiments, the subject is a human.

In certain embodiments, the neurological disorder is Alzheimer's disease, Parkinson's disease, Huntington's disease, amyotrophic later sclerosis (ALS), multiple sclerosis, dementia, traumatic brain injury, or stroke. In certain such embodiments, the neurological disorder is Alzheimer's disease.

In certain embodiments, the EVs are administered by injection, intranasal delivery, convection-enhanced delivery, oral administration, or infusion. In certain such embodiments, the EVs are administered by intranasal delivery.

In certain embodiments, the EVs are administered in a dose range of about 1×10⁹ to about 1×10¹⁴ vesicles per kg of body weight of the subject. In certain such embodiments, the EVs are administered in a dose of about 1×10¹⁰ to about 1×10¹³, about 1×10¹¹ to about 1×10¹², or about 6.5×10¹¹ vesicles per kg of body weight of the subject.

In certain embodiments, the administration is repeated over a period of time to provide sustained therapeutic benefit to the subject.

In certain embodiments, the EVs are modified to increase their stability in vivo.

In certain embodiments, the EVs are modified to increase their targeting to the brain. In certain such embodiments, the EVs are modified by attaching a targeting moiety.

In certain embodiments, the EVs are modified to increase their therapeutic efficacy.

In certain embodiments, the EVs comprise a therapeutic agent.

In certain embodiments, the EVs are administered in combination with another therapeutic compound. In certain such embodiments, the other therapeutic compound is a cholinesterase inhibitor, a memantine, an acetylcholine agonist, or a gamma-secretase inhibitor.

The present invention also relates in part to a composition comprising extracellular vesicles (EVs) derived from human induced pluripotent stem cell derived neural stem cells (hiPSC-NSCs).

In certain embodiments, the composition further comprising a carrier. In certain such embodiments, the carrier comprises a lipid or a polymer.

The present invention further relates in part to a kit for use in treating a neurological disorder comprising: a plurality of EVs derived from hiPSC-NSCs; and instructions for administering the EVs to a subject.

### BRIEF DESCRIPTION OF THE DRAWINGS

FIG. 1 illustrates that human neural stem cells (hNSCs) express specific markers and extracellular vesicles isolated from hNSC cultures express multiple EV markers.

FIG. 2 depicts microglial progenitors and mature microglia derived from hiPSCs.

FIG. 3 depicts the modulated expression of genes linked to activation of microglia, disease-associated microglia and proinflammatory signaling following treatment with hiPSC-NSC-EVs.

FIG. 4 illustrates brain regions showing incorporation of PKH26-labeled hiPSC-NSC-EVs into NeuN+ neurons in different regions of the forebrain, 45 minutes and 6 hours post administration.

FIG. 5 shows that intranasally administered PKH26-labeled hiPSC-NSC-EVs (red particles) that incorporated into neurons maintained CD63 expression.

FIG. 6 illustrated brain regions incorporating PKH26-labeled hiPSC-NSC-EVs into IBA-1+ microglia in different regions of the forebrain, 45 minutes and 6 hours post administration.

FIG. 7 depicts transcriptomic changes following incorporation of intranasally administered hiPSC-NSC-EVs into microglia.

FIG. 8 depicts modulation of the expression of disease-associate microglia (DAM) genes and genes linked to NLRP3 inflammasomes following intranasal treatment of hiPSC-NSC-EVs in 5XFAD mouse.

FIG. 9 depicts the results of an objection location test, showing that hiPSC-NSC-EV treatment maintains hippocampus-dependent cognitive function in 5XFAD mice.

FIG. 10 depicts the results of a pattern separation test, showing that hiPSC-NSC-EV treatment preserves dentate gyrus-dependent cognitive function in 5XFAD mice.

FIG. 11 depicts the results of a sucrose preference test, showing that hiPSC-NSC-EV treatment prevents anhedonia in 5XFAD mice.

FIG. 12 depicts reductions in activated microglial clusters in 5XFAD mice at 8 weeks after intranasal administration of hiPSC-NSC-EVs.

FIG. 13 depicts modulation of the expression of disease-associated microglia (DAM) genes in 5XFAD mice at 8 weeks after intranasal administration of hiPSC-NSC-EVs.

FIG. 14 depicts modulation of the expression of genes linked to NLRP3 inflammasome activation in the hippocampus of 5XFAD mice at 8 weeks after intranasal hiPSC-NSC-EV treatment.

FIG. 15 depicts inhibition of NLRP3 inflammasome activation in microglia in the hippocampus of 5XFAD mice at 8 weeks after intranasal hiPSC-NSC-EV treatment.

FIG. 16 depicts the inhibition of inflammatory MAPK signaling pathway proteins in the hippocampus of 5XFAD mice at 8 weeks following intranasal hiPSC-NSC-EV treatment.

FIG. 17 depicts changes in reactive astrocytes in 5XFAD mice at 8 weeks after intranasal administration of hiPSC-NSC-EVs.

FIG. 18 depicts changes in changes in amyloid plaques, and concentrations of Aβ42 and phosphorylated-tau proteins levels at 8 weeks after intranasal administration of hiPSC-NSC-EVs.

FIG. 19 depicts preservation of hippocampal neurogenesis in 5XFAD mice at 8 weeks after intranasal administration of hiPSC-NSC-EVs.

### DETAILED DESCRIPTION OF THE INVENTION

The following description and examples illustrate embodiments of the present disclosure in detail. It is to be understood that the present disclosure is not limited to the particular embodiments described herein and as such can vary. Those of skill in the art will recognize that there are variations and modifications of the present disclosure, which are encompassed within the scope of the present invention.

Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. The terminology used herein is for the purpose of describing particular embodiments only, and is not intended to be limiting.

In this application, the use of the singular includes the plural unless specifically stated otherwise. As used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

A “therapeutically effective amount” or “therapeutically effective dose” refers to an amount or dose effective, for periods of time necessary, to achieve a desired therapeutic result. The amount can vary according to factors such as the disease state, age, sex, and weight of the individual, and the ability of the inventive nucleic acid sequences to elicit a desired response in the individual.

“Polynucleotide” or “oligonucleotide” refers to a polymeric form of nucleotides or nucleic acids of any length, either ribonucleotides or deoxyribonucleotides. This term refers only to the primary structure of the molecule. Thus, this term includes double and single stranded deoxyribonucleic acid (DNA), triplex DNA, as well as double and single stranded ribonucleic acid (RNA). It also includes modified, for example, by methylation and/or by capping, and unmodified forms of the polynucleotide. The term is also meant to include molecules that include non-naturally occurring or synthetic nucleotides as well as nucleotide analogs.

“Polypeptide,” “peptide,” and their grammatical equivalents as used herein refer to a polymer of amino acid residues. The polypeptide can optionally include glycosylation or other modifications typical for a given protein in a given cellular environment. Polypeptides and proteins disclosed herein (including functional fragments and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenyl serine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides or proteins described herein in an engineered cell can be associated with post-translational modifications of one or more amino acids of the polypeptide or protein. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The term “isolated” and its grammatical equivalents as used herein refer to the removal of a nucleic acid, protein, vesicle, or cell from its natural environment. It is to be understood, however, that nucleic acids, proteins, vesicles, or cells can be formulated with diluents or adjuvants and still for practical purposes be isolated.

The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” For example, nucleic acids typically are mixed with an acceptable carrier or diluent when used for introduction into cells. The term “substantially purified” and its grammatical equivalents as used herein refer to a nucleic acid sequence, polypeptide, protein or other compound that is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Patient” or “subject” refers to a mammalian subject diagnosed with or suspected of having or developing an itch-related disorder such as atopic dermatitis. In some embodiments, the term “patient” refers to a mammalian subject with a higher than average likelihood of developing such a disorder. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” means a patient diagnosed with or suspected of having a disease or disorder, for instance, but not restricted to itch-related disorders.

“Administering” refers to herein as providing one or more compositions described herein to a patient or a subject. One or more routes of administration can be employed.

As used herein, the terms “treatment,” “treating,” and their grammatical equivalents refer to obtaining a desired pharmacologic and/or physiologic effect. In some embodiments, the effect is therapeutic, i.e., the effect partially or completely cures a disease and/or adverse symptom attributable to the disease. In some embodiments, the term “treating” can include “preventing” a disease or a condition.

As used herein, a “treatment interval” refers to a treatment cycle, for example, a course of administration of a therapeutic agent that can be repeated, e.g., on a regular schedule. In some embodiments, a dosage regimen can have one or more periods of no administration of the therapeutic agent in between treatment intervals.

The terms “administered in combination,” “co-administration,” “co-administering,” and “co-providing” as used herein, mean that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the first treatment and second treatment can be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment. The order of administration of the first and secondary treatment can also be reversed.

The terms “therapeutically effective amount,” therapeutic amount,” “immunogenically effective amount,” and their grammatical equivalents refer to an amount effective, at dosages and for periods of time necessary, to achieve a desired therapeutic or immunogenic result. The effective amount can vary according to factors such as the disease state, age, sex, and weight of the subject, and the ability of a composition described herein to elicit a desired response in one or more subjects. The precise amount of the compositions of the present disclosure to be administered can be determined by a physician or veterinarian with consideration of the age, weight, and condition of the patient (subject).

As used herein, terms used in the identification of biological moieties may include, or may not include, a dash “-” within the term. The presence or absence of a dash does not change the intended meaning or identification of the biological moiety.

The present invention provides a method for treating a neurological disorder, comprising administering a therapeutically effective amount of extracellular vesicles (EVs) derived from human induced pluripotent stem cell derived neural stem cells (hiPSC-NSCs) to a subject in need thereof.

In certain embodiments, the subject is a patient.

In certain embodiments, the subject is a mammal, for example, a primate. In some embodiments, the subject is a human.

In certain embodiments, the subject has recently been diagnosed with the neurological disorder.

In certain embodiments, the subject is in the mid-stage of the neurological disorder.

Examples of neurological disorders that may be treated using the method of the present invention include Alzheimer's disease, Parkinson's disease, Huntington's disease, amyotrophic: later sclerosis (ALS), multiple sclerosis, dementia, traumatic brain injury, or stroke.

In certain embodiments, the disorder to be treated is Alzheimer's disease. In certain embodiments, the disorder is moderate Alzheimer's disease. In certain embodiments, the disorder is severe Alzheimer's disease.

The EVs may be administered by any means recognized in the art for such delivery, for example by injection, intranasal delivery, convection-enhanced delivery, oral administration or infusion. The method of administration may be chosen based on factors such as the subject's age, the subject's overall health, and the severity of the disease.

In certain embodiments, the EVs are delivered by injection, for example to the brain. Such may, for example, be accomplished by intracerebroventricular (ICV) injection or subcutaneous injection.

In certain embodiments, the EVs are delivered by inhalation. Such delivery has the advantage of being less invasive and having a reduced risk of infection than injection or infusion and allows for a more efficient and targeted delivery than oral administration. In addition, inhalation allows for delivery of the EVs to the lungs which have a large surface area that is in direct contact with the bloodstream.

For delivery by inhalation, the EVs may be contained in a liquid formulation that is aerosolized into small particles that can be inhaled by the lungs. Such a formulation may be delivered, for example, by way of a nebulizer, inhaler, or a nasal spray.

Administration may be at any suitable site on the subject. The choice of administration site will vary depending on factors such as the volume of the dose to be administered, the subject's age, the subject's sex, and the type of active agent to be administered. Subcutaneous administration may, for example, be to the subject's limbs (e.g., arms, hands, fingers, legs, feet, and/or toes), buttocks, and/or abdomen. For doses having larger volumes, intramuscular administration is preferred. Such may be, for example, to the subject's deltoid, vastus lateralis, ventrogluteal, or dorsogluteal muscle. Intravenous administration may, for example, be to the subject's arm (e.g. at the bend of the arm), the back of the subject's hand, or the top of the subject's foot. Intra-articular administration may, for example, be to the subject's knee, hip, shoulder, or ankle.

In certain embodiments, the EVs are administered in a dose range of about 1×10⁹ to about 1×10¹⁴ vesicles per kg of body weight of the subject. In certain such embodiments, the EVs are administered in a dose of about 1×10¹⁰ to about 1×10¹³, about 1×10¹¹ to about 1×10¹², or about 6.5×10¹¹ vesicles per kg of body weight of the subject. The effective amount of extracellular vesicles to be administered may vary depending on the severity of the disease, age, gender, weight, and overall health of the subject. The amount may also vary depending on the condition to be treated, the EVs used for treatment, and the route of administration.

The dose may be adjusted during the course of treatment, for example, after the subject's condition is monitored.

The dosing regimen will vary depending on the subject's age, the subject's sex, and the type of active agent to be administered. The dose may be administered hourly, daily, weekly, monthly, or annually.

In certain embodiments, the doses are delivered at intervals at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days apart. In certain embodiments, the doses are delivered at intervals of about twice per day, about once every day, about twice per week, about once every week, about once every two weeks, about once every three weeks, about once every four weeks, or about once every five weeks. In certain embodiments, the second dose is administered about one week after the first dose, about two weeks after the first dose, about three weeks after the first dose, about four weeks after the first dose, or about five weeks after the first dose; the third dose is administered two weeks after the second dose, about three weeks after the second dose, about four weeks after the second dose, about five weeks after the second dose, or about six weeks after the second dose; and the fourth dose is administered about three weeks after the third dose, about four weeks after the third dose, about five weeks after the third dose, about six weeks after the third dose, about seven weeks after the third dose, about eight weeks after the third dose, about nine weeks after the third dose, about ten weeks after the third dose, about eleven weeks after the third dose, or about twelve weeks after the third dose. In one embodiment, the second dose is administered about two weeks after the first dose, the third dose is administered about six weeks after the second dose, and the fourth dose is administered about twelve weeks after the third dose.

The specific initial and continuing dosage regimen for each patient will vary according to the nature and severity of the condition as determined by the attending diagnostician, the activity of the therapeutic agent, the age of the patient, the diet of the patient, time of administration, route of administration, rate of excretion of the drug, drug combinations, and the like.

In certain embodiments, the administration of the EVs is repeated over a period of time to provide sustained therapeutic benefit to the subject. For example, such administration may be once per week for more than 5 weeks, 1-5 weeks, 1-4 weeks, 1-3 weeks, or 2 weeks. In another example, administration may be twice per week, for example, such administration may be once per week for more than 5 weeks, 1-5 weeks, 1-4 weeks, 1-3 weeks, or 2 weeks.

The desired mode of treatment, number of doses, routes of administration, and dose schedules may be ascertained and/or adjusted in accordance with methodologies known in the art.

The present invention also relates to a composition comprising extracellular vesicles (EVs) derived from human induced pluripotent stem cell derived neural stem cells (hiPSC-NSCs).

In certain embodiments, the composition further comprises a pharmaceutically-acceptable carrier.

In certain embodiments, the composition is for use in treating a neurological disorder, such as those described previously.

Proper formulation of the pharmaceutical composition is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Any suitable carrier can be used within the context of the present disclosure, and such carriers are well known in the art. The choice of carrier will be determined, in part, by the particular use of the composition (e.g., administration to an animal) and the particular method used to administer the composition. The pharmaceutical composition optionally can be sterile.

Suitable pharmaceutical compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The pharmaceutical composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution.

In some embodiments, the pharmaceutical composition is formulated to protect the EVs from damage prior to administration. For example, the pharmaceutical composition can be formulated to reduce loss of the EVs on devices used to prepare, store, or administer the EVs, such as glassware, syringes, or needles. The pharmaceutical composition can be formulated to decrease the light sensitivity and/or temperature sensitivity of the EVs. To this end, the pharmaceutical composition preferably comprises a pharmaceutically-acceptable liquid carrier, such as, for example, those described above, and a stabilizing agent selected from the group consisting of polysorbate 80, L-arginine, polyvinylpyrrolidone, trehalose, and combinations thereof.

In certain embodiments, the pharmaceutical composition may include one or more pH adjusting agents or buffering agents, including: acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the pharmaceutical composition in an acceptable range.

In certain embodiments, the pharmaceutical composition may comprise one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical composition may be formulated into any suitable dosage form, including but not limited to, aqueous oral dispersions, liquids, gels, syrups, elixirs, slurries, suspensions and the like. In some embodiments, the pharmaceutical compositions are formulated into solutions (for example, for IV or IN administration). In some cases, the pharmaceutical composition is formulated as an infusion. In some cases, the pharmaceutical composition is formulated as an injection. In some cases, the pharmaceutical composition is formulated as a solution that can be aerosolized for use in an intranasal spray.

In certain embodiments, the pharmaceutical composition is a liquid. In some embodiments, the composition may be lyophilized and then reconstituted before use.

In certain embodiments, the pharmaceutical composition may include one or more preservatives, for example, to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, the pharmaceutical composition may include one or more antifoaming agents. Antifoaming agents can reduce foaming during processing which can result in coagulation of aqueous dispersions, bubbles in the finished film, or generally impair processing. Exemplary anti-foaming agents include silicon emulsions or sorbitan sesquoleate.

In certain embodiments, the pharmaceutical composition may include one or more antioxidants. Exemplary antioxidants include butylated hydroxytoluene (BHT), sodium ascorbate, ascorbic acid, sodium metabisulfite and tocopherol. In certain embodiments, the one or more antioxidants enhance chemical stability of the composition.

In certain embodiments, the pharmaceutical composition may include one or more stabilizing agents. Exemplary stabilizing agents include, for example: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In certain embodiments, the pharmaceutical composition may include one or more binders. Binders can impart cohesive qualities. Exemplary binders include: alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

In certain embodiments, the pharmaceutical composition may include a carrier or a pharmaceutically-compatible carrier material. These may include any commonly used excipients in pharmaceutics and should be selected on the basis of compatibility with the pharmaceutical compounds described herein. Exemplary carrier materials include binders, suspending agents, disintegration agents, filling agents, surfactants, solubilizers, stabilizers, lubricants, wetting agents, diluents, and the like. Exemplary pharmaceutically-compatible carrier materials may include acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition may include one or more diffusion facilitating agents, dispersing agents, and/or viscosity modulating agents, for example, to control the diffusion and homogeneity of the composition through liquid media or a granulation or blend method. In some embodiments, these agents also facilitate the effectiveness of a coating or eroding matrix. Exemplary diffusion facilitators and dispersing agents may include hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68 ®, F88 ®, and F108 ®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

In certain embodiments the pharmaceutical composition comprises a combination of one or more erosion facilitators with one or more diffusion facilitators.

In certain embodiments, the pharmaceutical composition may include one or more diluents. A diluent is a chemical compound that is used to dilute the substance of interest prior to delivery. Diluents can also be used to stabilize substances because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the pharmaceutical composition may include one or more filling agents. Filling agents may include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the pharmaceutical composition may include one or more lubricants or glidants. These are compounds that prevent, reduce, or inhibit adhesion or friction of materials. Exemplary lubricants may include stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

In certain embodiments, the pharmaceutical composition may include one or more plasticizers. These are compounds used to soften the microencapsulation material or film coatings to make them less brittle. Exemplary plasticizers may include polyethylene glycols such as PEG 300, PEG 400, PEG 600, PEG 1450, PEG 3350, and PEG 800, stearic acid, propylene glycol, oleic acid, triethyl cellulose and triacetin. In some embodiments, the plasticizers may also function as dispersing agents or wetting agents.

In certain embodiments, the pharmaceutical composition may include one or more solubilizers. Exemplary solubilizers may include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In certain embodiments, the pharmaceutical composition may include one or more stabilizers. Exemplary stabilizers may include any antioxidation agents, buffers, acids, preservatives and the like.

In certain embodiments, the pharmaceutical composition may include one or more suspending agents. Exemplary suspending agents may include compounds such as polyvinylpyrrolidone (e.g., polyvinylpyrrolidone K12, polyvinylpyrrolidone K17, polyvinylpyrrolidone K25, or polyvinylpyrrolidone K30), vinyl pyrrolidone/vinyl acetate copolymer (S630), polyethylene glycol (e.g., the polyethylene glycol can have a molecular weight of about 300 to about 6000, or about 3350 to about 4000, or about 7000 to about 5400), sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics (e.g., sodium carboxymethylcellulose, methylcellulose, sodium carboxymethylcellulose, hydroxypropylmethylcellulose, hydroxyethylcellulose), polysorbate-80, sodium alginate, polyethoxylated sorbitan monolaurate, polyethoxylated sorbitan monolaurate, povidone and the like.

In certain embodiments, the pharmaceutical composition may include one or more surfactants. Exemplary surfactants may include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, the surfactants can be included in the pharmaceutical composition to enhance physical stability or for other purposes.

In certain embodiments, the pharmaceutical composition may include one or more viscosity enhancing agents. Exemplary viscosity enhancing agents may include methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

In certain embodiments, the pharmaceutical composition may include one or more wetting agents. Exemplary wetting agents may include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In certain embodiments, the pharmaceutical composition may be manufactured in a conventional manner, such as by means of conventional mixing, dissolving, granulating, dragee-making, levigating, emulsifying, encapsulating, entrapping, or compression processes.

In certain embodiments, the pharmaceutical composition described herein may conveniently be presented in unit dosage form and be prepared by any of the methods well known in the art of pharmacy. In general, the pharmaceutical compositions may be prepared by bringing the active ingredient into association with a carrier, and then, if necessary, shaping the product into the desired formulation. In the pharmaceutical composition, the EVs described herein is included in an amount sufficient to produce the desired effect upon the process, condition or disease sought to be treated.

In certain embodiments, the pharmaceutical composition comprises the EVs described herein in a therapeutically-effective amount.

In some embodiments, the pharmaceutical composition may be stored by freezing at a temperature of about 0° C. to about −120° C., about −10° C. to about −110° C., about −20° C. to about −100° C., about −30° C. to about −90° C., about −40° C. to about −90° C., about −50° C. to about −90° C., about −60° C. to about −90° C., about −65° C. to about −85° C., or about −70° C. to about −80° C. In some embodiments, the pharmaceutical composition may be stored by freezing at a temperature of about −60° C., about −61° C., about −62° C., about −63° C., about −64° C., about −65° C., about −66° C., about −66° C., about −67° C., about −68° C., about −69° C., about −70° C., about −71° C., about −72° C., about −73° C., about −74° C., about −75° C., about −76° C., about −77° C., about −78° C., about −79° C., about −80° C., about −81° C., about −82° C., about −83° C., about −84° C., about −85° C., about −86° C., about −87° C., about −88° C., about −89° C., or about −90° C. The pharmaceutical composition may be thawed, for example, in a water bath prior to use, avoiding prolonged exposure of the thawed composition to the water bath. The temperature of water bath used to thaw the pharmaceutical composition may be, for example, between about 30° C. to about 44° C., about 31° C. to about 43° C., about 32° C. to about 42° C., about 33° C. to about 41° C., about 34° C. to about 40° C., or about 35° C. to about 39° C. In some embodiments, the temperature of water bath used to thaw the pharmaceutical composition may be about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. The thawed composition may be stored for up to about 15 minutes, up to about 30 minutes, up to about 45 minutes, up to about 1 hour, up to about 75 minutes, up to about 90 minutes, up to about 105 minutes, or up to about 2 hours at ambient temperature prior to administration. In some embodiments, the thawed composition will appear as a clear to slightly opalescent, colorless liquid and be substantially free of visible particulates.

In certain embodiments, the method of treatment of the present invention involves the administration of the aforementioned composition.

The present invention also relates to the use of extracellular vesicles (EVs) derived from human induced pluripotent stem cell derived neural stem cells (hiPSC-NSCs), or a composition comprising such EVs, for the manufacture of a medicament for treating a neurological disorder, such as those described previously.

The present invention also relates to a kit or article of manufacture for use in in treating a neurological disorder. Suitable kits or articles of manufacture may comprise a plurality of EVs derived from hiPSC-NSCs. Such a kit or article of manufacture may include a package or container that comprises such EVs. Suitable containers include, for example, bottles, vials, syringes, and test tubes. In some embodiments, the containers are formed from a variety of materials, such as glass or plastic. Suitable articles of manufacture may contain packaging materials. Examples of pharmaceutical packaging materials include blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions may also be included. In certain embodiments, the instructions are for administering the EVs to a subject. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

In some embodiments, the kit contains a vial containing the composition of the present invention. In some such embodiments, the vial comprises, for example, from about 0.1 to about 20 ml of the pharmaceutical composition, from about 0.1 to about 15 ml of the pharmaceutical composition, from about 0.1 to about 10 ml of the pharmaceutical composition, from about 0.1 to about 9 ml of the pharmaceutical composition, from about 0.1 to about 8 ml of the pharmaceutical composition, from about 0.1 to about 7 ml of the pharmaceutical composition, from about 0.1 to about 6 ml of the pharmaceutical composition, from about 0.1 to about 5 ml of the pharmaceutical composition, from about 0.2 to about 5 ml of the pharmaceutical composition, from about 0.2 to about 4 ml of the pharmaceutical composition, from about 0.2 to about 3 ml of the pharmaceutical composition, from about 0.2 to about 2 ml of the pharmaceutical composition, from about 0.2 to about 1 ml of the pharmaceutical composition, from about 0.5 to about 2 ml of the pharmaceutical composition, from about 0.5 to about 1.75 ml of the pharmaceutical composition, from about 0.5 to about 1.5 ml of the pharmaceutical composition, from about 0.5 to about 1.25 ml of the pharmaceutical composition, from about 0.5 to about 1 ml of the pharmaceutical composition, from about 0.75 to about 1.25 ml of the pharmaceutical composition. In other such embodiments, the vial comprises, for example, about 0.5 ml of the pharmaceutical composition, about 0.55 ml of the pharmaceutical composition, about 0.6 ml of the pharmaceutical composition, about 0.65 ml of the pharmaceutical composition, about 0.7 ml of the pharmaceutical composition, about 0.75 ml of the pharmaceutical composition, about 0.8 ml of the pharmaceutical composition, about 0.85 ml of the pharmaceutical composition, about 0.9 ml of the pharmaceutical composition, about 0.95 ml of the pharmaceutical composition, about 1 ml of the pharmaceutical composition, about 1.05 ml of the pharmaceutical composition, about 1.1 ml of the pharmaceutical composition, about 1.15 ml of the pharmaceutical composition, or about 1.2 ml of the pharmaceutical composition.

The hiPSC-NSCs may be produced through any means known in the art for such culturing. Such methods are described, for example, in Upadhya et al., Journal of Extracellular Vesicles (2020), 9 (“Upadhya”), which is incorporated herein in its entirety. For example, hiPSCs may be grown in a neural induction medium containing neurobasal and neural induction supplement to induce the hiPSCs to differentiate into neural stem cells (NSCs). Following differentiation into NSCs, the cells may be broken up into individual cells (dissociated) and cultured

The EVs can be isolated from the hiPSC-NSCs through any means known in the art. Such methods are described, for example, in Upadhya. For example, the EVs may be isolated from a cell culture medium using standard techniques such as centrifugation, anion-exchange chromatography (AEC), size-exclusion chromatography (SEC), and/or polymer-based precipitation.

In certain embodiments, the EVs are isolated a combination of anion-exchange chromatography (AEC) and size-exclusion chromatography (SEC). In AEC, the EVs, which are negatively charged, bind to the column. The EVs are then eluted and collected. To further purify the EVs, SEC is performed the eluate from the AEC. SEC involves a column containing beads and works by separating molecules based on size. EVs are small and thus pass through the beads while the larger molecules cannot pass through the beads and end up being trapped in the column.

In certain embodiments, the identity and purity of the EVs are confirmed. The EVs can, for example, be characterized by electron microscopy, nanoparticle tracking analysis (NTA), ELISA, and/or Western blot analysis. NTA may, for example, be used to analyze the final concentration and size distribution of particles. ELISA and Western blotting may be used to determine the expression of common EV markers, such as CD63, CD9, and ALIX.

In certain embodiments, the protein and lipid contends of the EV preparations may be measured. The total protein content may be measured, for example, using a bicinchoninic acid (BCA) protein assay kit. The total lipid content may, for example, be measured using the modified sulpho-phospho vanillin (SPV) method, which involves sonication, vortexing, and incubation with sulfuric acid and phospho-vanillin reagent.

In certain embodiments, the identity and quantity of small RNAs, such as miRNAs, in the EV preparation may be determined. The RNA may, for example, be isolated using a mirVana miRNA Isolation kit. Quantification may be done using a RiboGreen Assay. Small RNA-sequencing data analysis may be performed using the Banana Slug analytics platform.

In certain embodiments, the presence of certain proteins of interest in the EVs is validated. Such may be done, for example, using ELISA.

In certain embodiments, the EVs are modified to allow for improved stability. Such may be done, for example, by encapsulating the EVs in a protective coating, for example one made of polymers, lipids (e.g., a liposome), and/or proteins. Such may also be done by adding stabilizing agents to the EVs, such as proteins, lipids, or other molecules such as trehalose, sucrose, polyvinyl alcohol, and polyethylene glycol (PEG). In certain embodiments, the surface of the EVs is modified, for example, by adding charged groups or modifying the hydrophobicity of the surface. In certain embodiments, the EVs are genetically engineered to express surface proteins or peptides that increase their stability or half-life, for example, CD47 which serves as a signal that prevents phagocytosis by macrophages.

In certain such embodiments, the modifications are made to allow for better stability of the EVs in vivo.

In certain embodiments of the present invention, the EVs are modified to increase their targeting to the brain. This may, for example, be done by attaching a targeting moiety. Examples of such a targeting moiety include an antibody or a peptide. The targeting agents may, for example, be attached to the surface of the EVs using standard conjugation techniques, such as carbodiimide or maleimide chemistry.

In certain embodiments, the EVs are modified to increase their therapeutic efficacy. For example, the EVs may be modified to contain a small molecule drug, a protein, and/or a nucleic acid.

In certain embodiments, the EVs comprise a therapeutic agent. Such may naturally be present in the EVs or the EVs may be modified to contain the same. Examples of such therapeutic agents include protein and nucleic acids. In certain embodiments, the nucleic acid is a siRNA, a miRNA, or a plasmid DNA.

In certain embodiments, the EVs comprise an miRNA, for example, miR23-5p, miR103a-3p, miR30a-3p, miR181a-5p, miR320a, miR 320b, miR26a-5p, or miR191-5p. In certain embodiments, the EVs comprise a protein, for example, pentraxin-3 (PTX3) and galectin-3 binding protein (Gal-3BP).

In certain embodiments, the EVs have a size ranging from about 1 to about 300 nm, for example from about 50 to about 200 nm.

The EVs may be stored at low temperatures. In certain embodiments, the EV-containing formulation is stored at below 0° C., for example below about −10° C., below about −15° C., below about −20° C., below about −25° C., or below about −30° C. In certain embodiments, the formulation is stored at about −10 to about −30° C. In certain embodiments, the formulation is stored at about −20° C. In certain embodiments, the formulation is cryopreserved at ultra-low temperatures such as about −80° C. or lower.

The present invention also relates in part to a combination therapy comprising the administration of the EVs described herein, or a composition comprising the same, and the concomitant administration of one or more additional compounds, molecules, compositions, or agents. The present invention also relates in part to a combination therapy comprising the administration of the EVs described herein, or a composition comprising the same, and the concomitant use of a surgical or non-surgical procedure.

In certain embodiments, the at least one additional therapy comprises the co-administration of an additional agent. In some embodiments, the additional agent may be contained in the same composition that contains the EVs herein. Such combination therapies may serve to enhance the treatment of a disease or disorder (e.g., improving the subject's response, prolonging the effects of the treatment) and/or to reduce any side-effects of treatment.

Any suitable agent that may be combined with EVs described herein, or a composition comprising the same, may be used.

In certain embodiments, the additional agent is administered at or near the same location as the composition comprising the EVs of the present invention is administered. In certain other embodiments, the additional agent is administered at a different location, for example, at the opposite side or extremity.

Administration of the additional agent may be simultaneous with the administration of the composition comprising the EVs of the present invention. In certain embodiments, the additional agent is contained in the same formulation as that containing the EVs and can be administered with the EVs in one unitary dose. In certain other embodiments, the additional agent is not contained in the same formulation but is administered at the same time or within a limited time frame (e.g., a single day, hour, or fraction of an hour) from the administration of the EVs.

Alternatively, administration of the additional agent may be sequential in relation to the administration of the EVs of the present invention. Such may be preferred in instances where minimizing adverse reactions is desired. In such embodiments, the additional agent may be administered on a schedule in accordance with approved dosing regimens for that agent. Alternatively, the agent may be administered in accordance with a schedule that serves to better maximize the therapeutic effects of the combination therapy, while minimizing adverse reactions.

In certain embodiments, the EVs of the present invention may first be administered to a subject for purposes of treating a disease or disorder and then, at a later date, an additional agent may be administered. In one embodiment, the additional agent is administered at a time when, following the administration of the EVs of the present invention, the disease or condition is deemed relapsed, progressed, or non-responsive to said EVs.

In certain embodiments, the method of the present invention can be used alone or in combination with other treatments for a neurological disorder, for example, agents that target the underlying mechanisms of the disease. In certain embodiments, the disorder is Alzheimer's disease, Parkinson's disease, Huntington's disease, amyotrophic later sclerosis (ALS), multiple sclerosis, dementia, traumatic brain injury, or stroke.

In certain embodiments, the method also involves the administration of another therapeutic compound. The therapeutic compound may be a compounds for use in treating the neurological disorder, for example, a cholinesterase inhibitor, a memantine, an acetylcholine agonist, or a gamma-secretase inhibitor.

In certain embodiments, the composition or kit of the present invention comprises the aforementioned therapeutic compound.

In certain embodiments, the disorder is Alzheimer's disease and the method involves treating the subject with metformin or amyloid-beta reducing drugs.

In certain embodiments; the disorder is Parkinson's disease and the method involves treating the subject with levodopa.

In certain embodiments, the disorder is multiple sclerosis and the method involves treating the subject with interferon-beta 1a.

In certain embodiments, the disorder is ALS and the method involves treating the subject with Riluzole®, Edaravone®, or sodium phenylbutyrate-taurursodiol.

In certain embodiments, the disorder is dementia and the method involves treating the subject with memantine.

In certain embodiments, the method also involves the administration of an immune modulator. Examples of such immune modulators include a cytokine, an antibody, or a T cell modulator.

In certain embodiments, the composition or kit of the present invention comprises the aforementioned immune modulator.

In certain embodiments, the method also involves the administration of a neurotrophic factor. Examples of such neurotropic factors include brain-derived neurotrophic factor (BDNF), nerve growth factor (NGF), or non-steroidal anti-inflammatory drugs (NSAIDs).

In certain embodiments, the composition or kit of the present invention comprises the aforementioned neurotropic factor.

In certain embodiments, the method also involves the administration of an antioxidant.

In certain embodiments, the composition or kit of the present invention comprises the aforementioned antioxidant.

In certain embodiments, the hiPSCs are obtained from a subject with a neurological disorder, for example, Alzheimer's disease, Parkinson's disease, Huntington's disease, amyotrophic later sclerosis (ALS), multiple sclerosis, dementia, traumatic brain injury, or stroke. In certain such embodiments, the hiPSCs are obtained from the same subject as that which will be treated by administrations of EVs derived from the hiPSC-NSCs.

The present invention provides a novel method for treating neurological disorders, such as Alzheimer's disease, Parkinson's disease, Huntington's disease, amyotrophic later sclerosis (ALS), multiple sclerosis, dementia, traumatic brain injury, or stroke. Extracellular vesicles released by hiPSC-derived NSCs can modulate the functions of recipient cells, promoting tissue repair and improving cognitive function. The method of the present invention is non-invasive, safe, and effective, and can be used alone or in combination with other treatments for the neurological disorder.

### EXAMPLES

### Example 1—Generation of NSCs from hiPSCs

Human induced pluripotent stem cell (hiPSC) colonies (IMR90-4; Wisconsin International Stem Cell Bank, Madison, WI, USA) were grown as cell clumps at a density of 2-2.5×10⁴ cells/cm² in six-well plates coated with matrigel (Corning, Tewksbury, MA, USA) using TeSR™-E8™ medium (STEMCELL Technologies, Vancouver, Canada). Twenty-four hours later, the culture medium was replaced with neural induction medium containing neurobasal (Gibco, Grand Island, New York, USA) and neural induction (Gibco) supplement to induce the hiPSCs to differentiate into neural stem cells (NSCs). The medium was swapped every other day for seven days.

The primitive NSCs were dissociated with accutase (Gibco) and plated on matrigel-coated dishes with a density of 0.5-1.0×10⁵ cells per cm² in an NSC expansion medium containing 50% neurobasal, 50% advanced DMEM/F12, and 1× neural induction supplement. The culture medium was exchanged every other day until NSCs reached confluency on day 5 of plating.

The NSC cultures were passaged every seven days, and NSCs from different passages were cryoprotected and stored in liquid nitrogen.

The NSC status at different passages was confirmed through immunofluorescence staining for nestin (anti-nestin, 1:1000; EMD Millipore, Burlington, MA, USA) and Sox-2 (anti-Sox-2, 1:300; Santacruz Biotechnology, Dallas, TX, USA).

### Example 2—Collection of hiPSC-NSC Culture Media for Harvesting EVs

Frozen vials containing passage 11 NSCs were thawed at 37° C. and plated on to a T-75 culture flask (Corning) and grown at 37° C. in a CO₂ incubator.

Following 70% confluency, the cells were dislodged using 1 U/ml of dispase (Gibco), washed with NSC media (Gibco), and seeded at ˜500 cells per cm² into 150×20 mm diameter tissue culture plates (Corning) in NSC expansion medium.

Once NSCs reached 90% confluency, the media was harvested and used for isolating EVs or stored at −80° C. for further use.

### Example 3—E Isolation by Ion Exchange and Size Exclusion Chromatography

The conditioned media containing EVs were subjected to low-speed centrifugation at 3,000 rpm for 10 minutes, which was followed by filtration through a 0.22 μm filter GE Healthcare, Uppsala Sweden) to remove the cell debris and larger suspending particles. The large volume of filtered media was then subjected to a 5-7 fold concentration using Amicon 30 kDa cut-off ultra-filtration device (Millipore, MA, USA). Using a 1.5×12 cm chromatography column (Bio-Rad, Hercules, CA, USA), 10 ml of Q-Sepharose fast flow (GE Healthcare, Chicago, Illinois, USA) was equilibrated with 100 ml of equilibration buffer, and the chromatography was performed by adding the concentrated conditioned media. The EVs were selectively eluted using elution buffer containing 50 mM Tris and 1000 mM NaCl of pH 8.0. Fractions were collected at a flow rate of 1 ml/minute, and elution of EVs was continuously monitored by Nanoparticle tracking analysis (NanoSight LM10, Malvern Panalytical, Malvern, UK).

After AEC, the fractions containing EVs were pooled and concentrated using an ultrafiltration device of 30 kDa cut-off and subjected to an SEC on a column made up of 25 ml of Sephacryl S-500 High Resolution (GE Healthcare, Uppsala, Sweden). Using the mobile phase containing 50 mM phosphate buffer and 200 mM NaCl of pH 7.0, EVs were size-fractionated, and fractions were collected at a flow rate of 1 ml/minute. The elution of EVs was continuously monitored by quantifying the total protein content by bicinchoninic acid (BCA) method and tracked by nanoparticle tracking analysis. Fractions containing a high number of EVs with less protein content were pooled, concentrated and stored at −20° C. for further use.

### Example 4—Confirmation of EV-Specific Proteins

The EVs obtained from NSC cultures were subjected to western blot or ELISA to confirm the presence of EV-specific proteins. The EVs expressed several EV-specific markers, such as CD63, ALIX, and CD9.

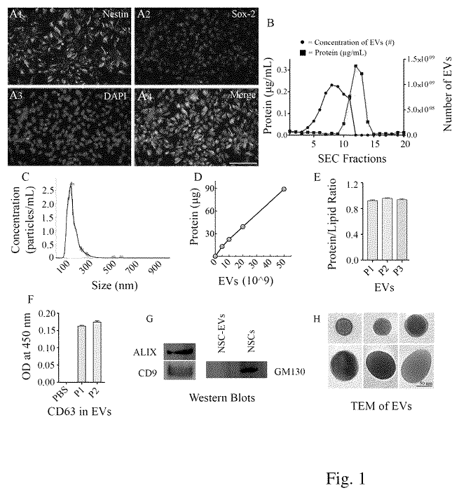

FIGS. 1 A1-A4 illustrate that all cells in the passage 11 hNSCs derived from human induced pluripotent stem cells (hiPSCs) express NSC markers nestin and Sox-2. Scale bar: 100 μm. FIG. 1B compares protein concentrations (the left Y-axis), and the concentration of EVs (the right Y-axis) in different EV fractions (X-axis) collected from size-exclusion chromatography (SEC). Note that fractions 5-9 contain most EVs with minimal protein content. FIG. 1C is a representative graph from NanoSight analysis showing the size of EVs. A linear relationship between the number of EVs and the total protein is shown in FIG. 1D. The protein-lipid ratio in EVs is shown in FIG. 1E. CD 63 protein content measured through ELISA is shown in FIG. 1F. FIG. 1G shows the presence of ALIX and CD9 in EVs evaluated through western blotting. The figure also indicates the absence of a deep cellular marker GM130 in hNSC-derived EVs, in contrast to its robust presence in the NSC lysate. FIG. 1H shows the EVs of different size and shape visualized through transmission electron microscopy. Scale bar, 50 nm.

### Example 5—Anti-Inflammatory Activity of hiPSC-NSC-EVs in Human Microglial Cultures Exposed to Amyloid Beta 42 (Aβ42)

Human microglia (iMicroglia) were generated from hiPSCs using methods from published protocols (Guttikonda et al., 24: 343-354, 2021; Upadhya et al., Frontiers in Molecular Neuroscience, 15: 845542, 2022). Immunocytochemistry confirmed the maturation of iMicroglia using antibodies against microglia-specific markers (Upadhya et al., Frontiers in Molecular Neuroscience, 15: 845542, 2022).

Mature iMicroglia cells were exposed to 1 μM Aβ42 oligomers for 24 hours.

Four hours after adding Aβ42, hiPSC-NSC-EVs (at a concentration of 40×10⁹ EVs) were added to subsets of iMicroglia cultures. Additional iMicroglia cultures receiving neither Aβ42 oligomer nor hiPSC-NSC-EVs were included as controls. Twenty-four hours later, iMicroglia were dissociated, and total RNA was isolated for quantitative real-time PCR (qRT-PCR studies). The RNA (500 ng/μL) samples were converted into cDNA using RT2 First Strand Kit (Qiagen). The qRT-PCR was performed using RT2 SYBR Green qPCR Mastermix and Primer mix (GeneCopoeia) for examining the expression of multiple genes linked to naïve and activated human microglia.

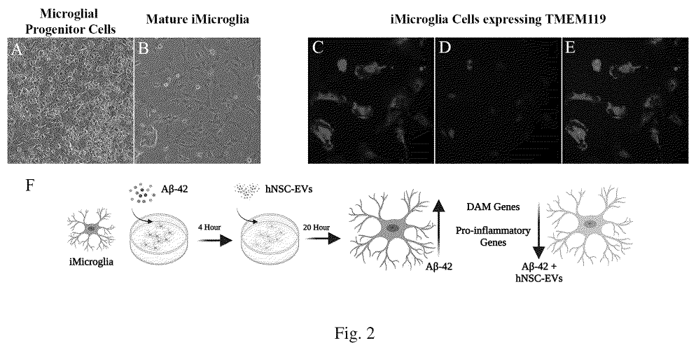

FIGS. 2A and B show the microglial progenitors and mature microglia derived from hiPSCs. The expression of TMEM119 confirmed the conversion of hiPSCs into mature microglia (FIGS. 2 C-E). The experimental design (FIG. 2F) depicts the activation of microglial cells with AP oligomers and treatment with hNSC-EVs.

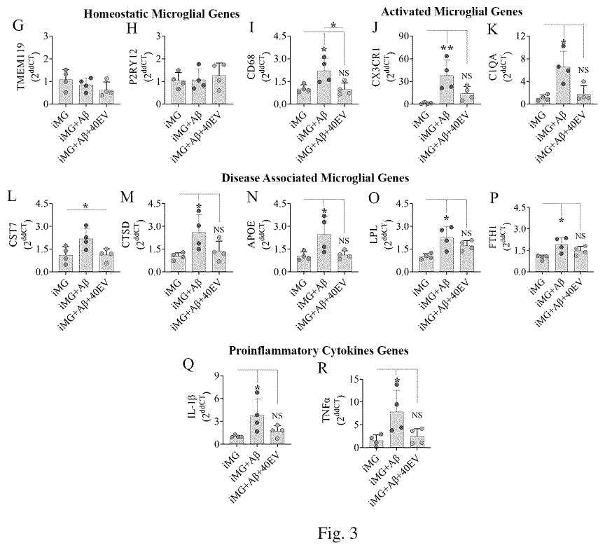

Homeostatic microglia genes (tmem119, p2ry12), activated microglia genes (cd68, cx3cr1, c1qa), disease-associated microglia (DAM) genes (cst7, ctsd, apoe, lpl, fth1) and proinflammatory genes (il1b, tnfa) were measured. The homeostatic microglia gene expression did not differ between groups (p>0.05; FIGS. 3G-H). The genes linked to microglial activation cd68, cx3cr1, and c1qa were significantly increased in Aβ42 exposed microglia (p<0.05-0.01; FIGS. 3I-K). Notably, hiPSC-NSC-EV treatment normalized the expression of many of the above genes to naive control levels (p>0.05; FIGS. 3I-K). DAM gene expression differed between groups. The genes such as ctsd, apoe, lpl, and fth1 displayed increased expression in the Aβ42 treated microglia (p<0.05; FIGS. 3M-P) but not in Aβ42 treated microglia receiving hiPSC-NSC-EVs (p>0.05). In addition, the expression of proinflammatory cytokine genes such as il1b and tnfa were increased in Aβ42 treated microglia (p<0.05; FIGS. 3Q-R) but were normalized to naive control levels in Aβ42 treated microglia receiving hiPSC-NSC-EVs (p>0.05).

hiPSC-NSC-EVs can modulate microglia exposed to Aβ42, one of the pathological proteins driving Alzheimer's disease pathogenesis through formation of amyloid plaques in the extracellular space. In this in vitro study, hiPSC-NSC-EVs reduced/normalized the expression of multiple genes linked to activation of microglia, disease-associated microglia and proinflammatory signaling.

### Example 6—Breeding and Maintenance of Mice Displaying Early-Onset Alzheimer's Disease

Both transgenic 5XFAD mice and their background (wild type) strain (B6SJLF1/J) were purchased from Jackson Labs (Cat No: 34840-JAX and 100012-JAX, Bar Harbor, Maine, USA) and maintained on B6/SJL genetic background by crossing 5XFAD transgenic male mice with B6SJLF1/J female mice. The required number of mice were bred and maintained at the Texas A&M University vivarium. Two groups of 5XFAD mice (AD mice receiving either vehicle or hiPSC-NSC-EVs, referred to as AD-Veh and AD-EV groups) and an age-matched naïve control group were included in all studies.

### Example 7—Intranasal (IN) Administration of hiPSC-NSC-EVs into 5XFAD Mice

In RNAseq and long-term studies, mice in AD-Veh and AD-EV groups received two IN doses of hiPSC-NSC-EVs (200×10⁹ EVs/dose, once weekly for two weeks) or vehicle (phosphate buffered saline). In the biodistribution study, AD mice and naïve mice received a single IN dose of hiPSC-NSC-EVs (25×10⁹ EVs). IN administrations of hiPSC-NSC-EVs were done as described in Upadhya et al., Journal of Extracellular Vesicles (2020), 9: 1809064, 2020.

### Example 8—Investigation of the Incorporation of Intranasally Administered hiPSC-NSC-EVs into Neurons and Microglia in 5XFAD Mice

AD mice and naïve mice were euthanized at 45 minutes or 6 hours (n=4/group/time point) after receiving a single intranasal administration of hiPSC-NSC-EVs. The brain tissues were examined for the incorporation of hiPSC-NSC-EVs into neurons and microglia in various regions of the forebrain, midbrain and hindbrain using dual and triple immunofluorescence and confocal microscopy methods.

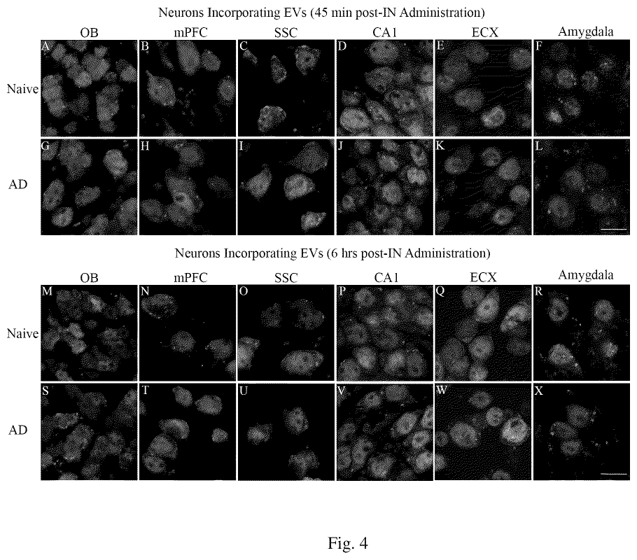

Intranasally administered hiPSC-NSC-EVs incorporated into cell bodies of neurons in different regions of forebrain, midbrain, and hindbrain in naive and AD mice. Investigation of PKH26+ structures in serial brain tissue sections processed for NeuN immunofluorescence using 0.5 μm thick Z-sections in a confocal microscope revealed widespread incorporation of IN administered hiPSC-NSC-EVs into neurons in the forebrain, midbrain, and hindbrain. hiPSC-NSC-EVs incorporated into −98% of neurons in both naïve and AD mice when examined at 45 minutes and 6 hours post-IN administration. Examples of neurons that incorporated PKH26-labeled EVs (red particles) at 45 minutes and 6 hours post-IN administration in different brain regions are illustrated in FIG. 4.

The illustrated brain regions include the olfactory bulb (OB; FIGS. 4A, G, M, S), medial prefrontal cortex (mPFC; FIGS. 4 B, H, N, T), the somatosensory cortex (SSC; FIGS. 4 C, I, O, U), CA1 subfield of the hippocampus (FIGS. 4 D, J, P, V), entorhinal cortex (ECX; FIGS. 4 K, Q, W), and amygdala (FIGS. 4 F, L, R, X), Quantification revealed that at 45 minutes and 6 hours post-IN administration, the percentage of neurons incorporating PKH26-labeled hiPSC-NSC-EVs in different regions of the forebrain, midbrain, and hindbrain were comparable between the naïve and AD groups.

Intranasal administration of 25×10⁹ hiPSC-NSC-EVs can target a vast majority of neurons in the entire brain of naïve and AD mice within 45 minutes.

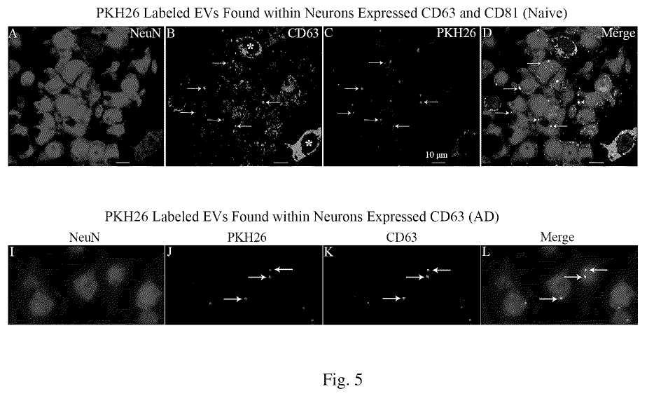

To confirm that the PKH26+ red particles found within and outside the soma of neurons are the intranasally administered hiPSC-NSC-EVs, careful Z-section analysis of brain tissue sections processed for NeuN and CD63 dual immunofluorescence was performed. Such analysis revealed that virtually all red particles found within neurons and outside neurons expressed both CD63. See FIG. 5.

Intranasally administered PKH26-labeled hiPSC-NSC-EVs (red particles) that incorporated into neurons maintained CD63 expression. Upper pane of FIG. 5, an example from naïve mouse. Lower panel of FIG. 5: an example from Alzheimer's disease mouse.

PKH26+ red particles found within neurons in the brain following intranasal administration of PKH26-labelled hiPSC-NSC-EVs are indeed EVs and not dye particles.

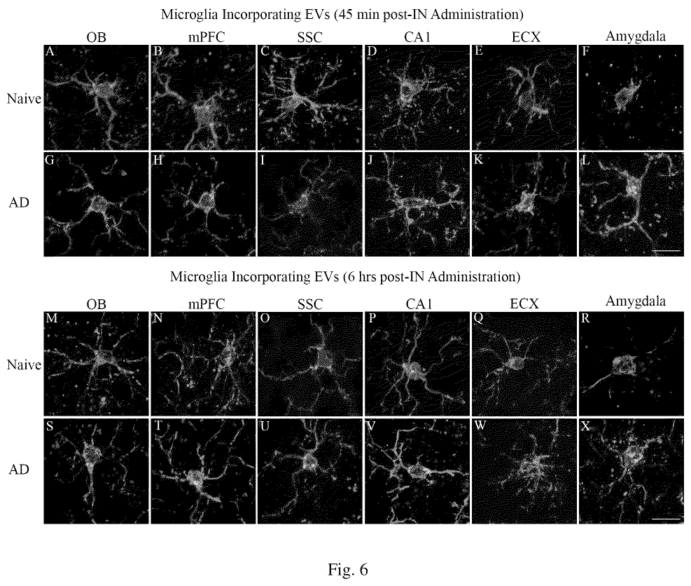

Examination of PKH26+ structures in serial brain tissue sections processed for IBA-1 immunofluorescence using 1.0 μm thick Z-sections in a confocal microscope revealed widespread accumulation of IN administered hiPSC-NSC-EVs into microglia in the forebrain, midbrain, and hindbrain. hiPSC-NSC-EVs accumulated inside the soma of ˜98% of microglia in both naïve and AD mice when examined at 45 minutes and 6 hours post-IN administration. Examples of microglia that accumulated PKH26-labeled EVs (red particle aggregates) within soma at 45 minutes and 6 hours post-IN administration in different brain regions are illustrated in FIG. 6.

The illustrated brain regions include the olfactory bulb (OB; FIGS. 6A,G,M,S), medial prefrontal cortex (mPFC; FIG. 6 B,H,N,T), somatosensory cortex (SSC; FIGS. 6 C,I,O,U), CA1 subfield of the hippocampus (FIGS. 6 D,J,P,V), entorhinal cortex (ECX; FIGS. 6 K,Q,W), and amygdala (FIGS. 6 F,L,R,X). Quantification revealed that at 45 minutes and 6 hours post-IN administration, the percentage of microglia incorporating PKH26-labeled hiPSC-NSC-EVs in different regions of the forebrain, midbrain and hindbrain were comparable between the naïve and AD groups.

Intranasal administration of 25×10⁹ hiPSC-NSC-EVs can target a vast majority of microglia in the entire brain of naïve and AD mice within 45 minutes.

### Example 9—Assessment of Transcriptomic Changes in Microglia of 5XFAD Mice Following Incorporation of Intranasally Administered hiPSC-NSC-EVs

AD mice receiving hiPSC-NSC-EVs, or the vehicle were euthanized 72 hours after the second intranasal dose. Microglia were quickly isolated from fresh brains and processed for sc-RNAseq study. Briefly, a single cell suspension of live microglia was obtained using gentleMACS™ Tissue Dissociator (Miltenyi Biotec) and MACS® Separator. Microglia were subjected to scRNA sequencing at Texas A&M Institute for Genome Sciences and Society. Individually barcoded libraries were pooled and sequenced on a NextSeq mid-output paired-end sequencing run at 2×75, using NextSeq 500/550 Mid-Output v2.5 kit (Illumina, San Diego, CA, USA) according to the manufacturer's instructions. The reads of scRNA libraries were aligned to the human GRCh38.p13 reference genome using Cell Ranger (version 7.0), and the resulting expression matrices were analyzed using scGEAToolbox.

Intranasally administered hiPSC-NSC-EVs induced transcriptomic changes following their incorporation into microglia in Alzheimer's mice.

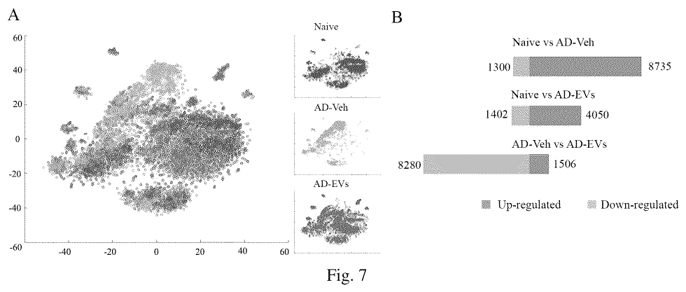

Gene expression patterns in the majority of microglia from AD mice receiving vehicle (AD-Veh group) were distinct from naïve mice (naïve) and AD mice receiving hiPSC-NSC-EVs (AD-EVs group) at 72 hours post-EV administration (FIG. 7A). The expression of 8,735 genes was upregulated, and 1300 genes were downregulated in the AD-Veh group compared to the naïve group. Whereas, in the AD-EV group, the expression of 4,050 genes was upregulated, 1402 genes were downregulated compared to the naïve group. Moreover, compared to the AD-Veh group, the AD-EV group displayed upregulation of 1,506 genes and downregulation of 8,280 genes (FIG. 7B), implying significant modulation of the expression of microglial genes by hiPSC-NSC-EVs.

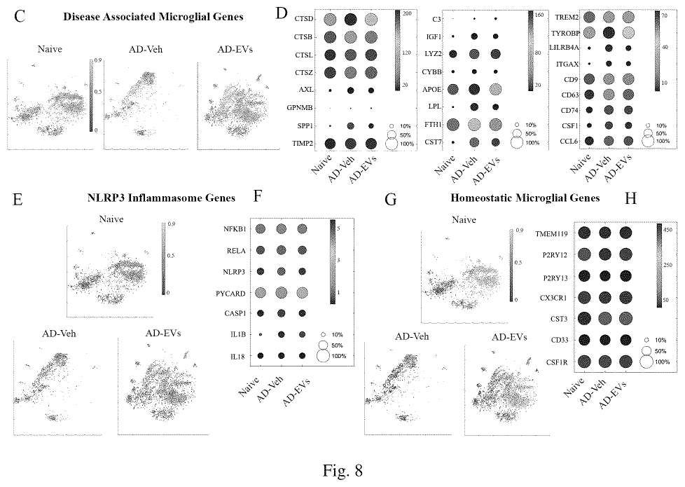

Disease-associated microglia (DAM) genes include ctsd, ctsb, ctsl, ctsz, axl, gpnmb, spp1, timp2, c3, igf1, lyz2, cybb, apoe, lpl, fth1, cst7, trem2, tyrobp, lilrb4a, itgax, cd9, cd63, cd74, csf1, cc16. These genes were upregulated the AD-Veh group compared to the naïve group (FIGS. 8 C, D). However, most of these genes, were downregulated in the AD-EVs group (FIGS. 8 C-D).

The expression of genes linked to NLRP3 inflammasomes, such as nfkb1, rela, nlrp3, pycard, casp1, il1b, il18 were upregulated in the AD-Veh group. However, the expression of many of these genes was reduced in the AD-EVs group (FIGS. 8 E-F). Furthermore, the expression of microglia homeostatic genes such as, P2RY12, P2RY13, CX3CR1, CD33 were downregulated in the AD-Veh group but upregulated in the AD-EVs group (FIGS. 8 G-H).

Intranasally administered hiPSC-NSC-EVs incorporate into microglia in Alzheimer's disease mice, which results in transcriptomic changes in microglia leading to the transformation of highly proinflammatory microglia into milder proinflammatory or non-inflammatory microglia at 72 hours post-EV administration. This is evidenced by reduced expression of genes linked to disease-associated microglia and NLRP3 inflammasomes, which are multiprotein complexes involved in the perpetuation of neuroinflammation in Alzheimer's brain.

### Example 10—Object Location Test (OLT)

This behavioral test examines the ability of animals to detect minor changes in their immediate environment. The proficiency of animals in this test is detected by their choice to explore the object moved to a new location in an arena. This function depends on the integrity of the dorsal hippocampus.

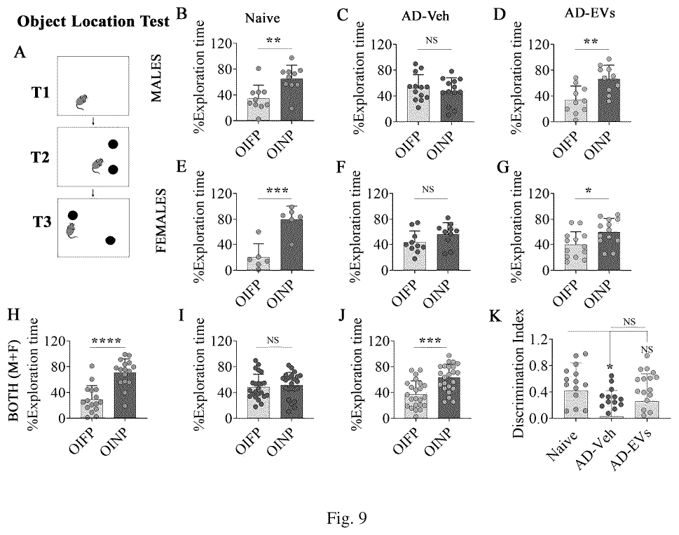

This test comprised 3 trials (T1-T3), with each trial lasting 5 minutes, and trials were separated by an inter-trail interval (ITI) of 15 minutes (FIG. 9A). T1 involved the exploration of an empty open field apparatus for habituation. T2 involved the exploration of two identical objects placed on one side of the open field apparatus. T3 involved the exploration of objects from T2 with one of the objects moved to a new location.

The results of the object learning test are shown in FIGS. 9B-H. Proficiency for discerning minor changes in the immediate environment (a cognitive function) was clearly evident in age-matched naïve male and female mice, as they preferred the object in the novel place (OINP) over the object in the familiar place (OIFP) (p<0.01-0.0001; FIGS. 9 B, E, H), but was impaired in both male and female AD mice receiving vehicle (p>0.05, FIGS. 9 C, F, I). However, male and female AD mice receiving hiPSC-NSC-EVs, akin to naïve mice, displayed proficiency for discerning minor changes in the immediate environment, as they preferred the OINP over the OIFP (p<0.05-0.001, FIGS. 9 D, G, J). When both genders were considered together, similar results were observed (FIGS. 9 H-J). Comparison of the OINP discrimination index revealed differences between groups (p<0.05, FIG. 9 K), with the AD-Veh group showing cognitive impairment.

AD mice receiving the vehicle displayed hippocampus-dependent cognitive impairment in an object location test. However, AD mice receiving hiPSC-NSC-EVs did not exhibit such impairment, implying that intranasal hiPSC-NSC-EV treatment in the early phase of AD can delay hippocampus-dependent cognitive impairment.

### Example 11—Pattern Separation Test (PST)

Pattern separation ability requires encoding of similar but not identical experiences in a non-overlapping fashion in the hippocampus. Proficiency in pattern separation can be measured through a PST. This function depends upon the integrity of the dentate gyrus of the hippocampus.

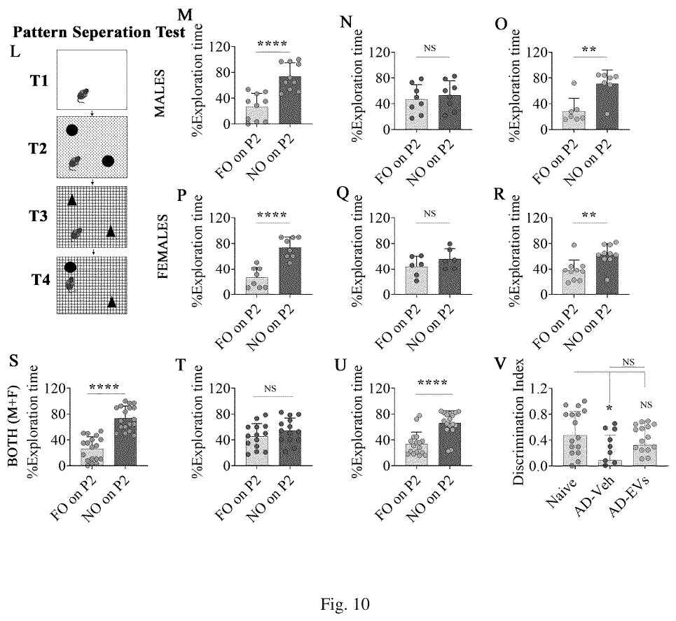

This test comprised 4 trials (T1-T4), with each trial lasting 5 minutes, and trials were separated by an inter-trail interval (ITI) of 60 minutes (FIG. 10L). T1 involved the exploration of an empty open field apparatus. T2 involved the exploration of a set of identical objects (type 1 objects) placed on a specific floor pattern (pattern 1; P1). T3 involved the exploration of a second set of identical objects (type 2 objects) placed on a different type of floor pattern (pattern 2; P2). T4 involved the exploration of two objects placed on P2, which included an object from T3 (i.e., a familiar object on P2; FO on P2) and an object from T2 (i.e., a novel object on P2; NO on P2). The choice to explore the NO on P2 over FO on P2 reflects the ability of animals to distinguish similar but not identical experiences in a non-overlapping fashion.

The results of the object learning test are shown in FIGS. 10M-V. Proficiency for pattern separation function (another cognitive function) was clearly evident in age-matched naïve male and female mice, as they preferred the novel object on pattern 2 (NO on P2) over the familiar object on pattern 2 (FO on P2) (p<0.0001, FIGS. 10 M, P, S), but was impaired in both male and female AD mice receiving vehicle (p>0.05, FIGS. 10 N, Q, T). However, male and female AD mice receiving hiPSC-NSC-EVs, akin to naïve mice, displayed proficiency for pattern separation, as they preferred NO on P2 over FO on P2 (p<0.01-0.0001, FIGS. 10 O, R, U). When both genders were considered together, similar results were observed (FIGS. 10 S-U). Comparison of the NO on the P2 discrimination index revealed differences between groups (p<0.05, FIG. 10y), with the AD-Veh group showing impaired pattern separation ability.

AD mice receiving the vehicle displayed impaired pattern separation ability. However, AD mice receiving hiPSC-NSC-EVs did not exhibit such impairment, implying that intranasal hiPSC-NSC-EV treatment in the early phase of AD can delay hippocampus-dependent pattern separation impairment.

### Example 12—Sucrose Preference Test

Anhedonia is a measure of depressive-like behavior. Anhedonia in individuals is typified by an inability to experience pleasure in activities that are otherwise pleasurable in healthy conditions. Anhedonia in AD mice was probed through a sucrose preference test (SPT), which involved an assessment of their preference to drink sweet water over standard water. A decreased preference for sweet water over standard water determined anhedonia.

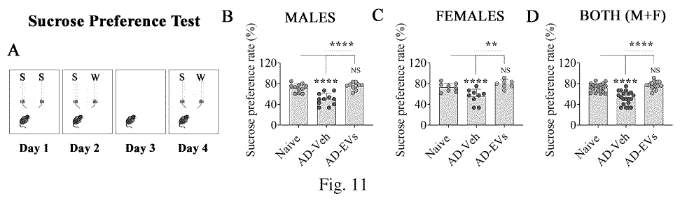

This test is performed 7 weeks after intranasal administration of hiPSC-NSC-EVs. On Day 1, the mice are given access to sucrose water, on Day 2, the mice are given access to both sucrose and standard water, on Day 3 the mice are fasted for 22 hours, and on Day 4 the sucrose preference test is performed with the mice are given access to both sucrose and standard water for 2 hours (FIG. 11A).

Male and female naïve mice demonstrated no anhedonia as they preferred sucrose-containing (sweet) water over standard water, which is evident from the analysis of sucrose preference rate (SPR) (FIGS. 11B-C). Male and female AD mice receiving the vehicle displayed anhedonia, as they did not exhibit a predilection for drinking sweet water (FIGS. 11 B-C). However, male and female AD mice receiving hiPSC-NSC-EVs, akin to naïve mice, displayed no anhedonia, as they preferred sweet water over standard water (FIGS. 11 B-C). Similar results were observed when both genders were considered together (FIG. 11D). Comparison of SPR across groups revealed significantly reduced SPR in both male and female AD mice receiving vehicle, compared to their naïve counterparts (p<0.0001). However, SPR was comparable between male and female naïve mice and male and female AD mice receiving hiPSC-NSC-EVs (p>0.05).

AD mice receiving the vehicle displayed anhedonia (depressive-like behavior). However, AD mice receiving hiPSC-NSC-EVs did not exhibit anhedonia, implying that intranasal hiPSC-NSC-EV treatment in the early phase of AD can delay mood impairment observed in the later phase of AD.

### Example 13—Changes in Activated and Disease-Associated Microglia

Following the aforementioned behavioral tests, an investigation of changes in activated and disease-associated microglia in the mice was conducted 8 weeks after intranasal administration. Subsets of animals from every group (n=6/group) were deeply anesthetized and euthanized through perfusion with a 4% paraformaldehyde. The fixed brain tissues were processed for immunohistochemical and immunofluorescence studies. Additional subsets of animals (n=6/group) were deeply anesthetized, and fresh brain tissues were harvested following decapitation. The fresh brain tissues were used for biochemical and molecular biological studies. The number of microglia via stereological counting of IBA1+ cells using every 20th section through the entire hippocampus. The number of microglial clusters per unit volume in the hippocampus was also quantified. Furthermore, the occurrence of NLRP3 inflammasomes (multiprotein complexes involved in the perpetuation of proinflammatory microglia) were examined through triple immunofluorescence for IBA-1, NLRP3, and apoptosis-associated speck-like protein containing a CARD (ASC) and confocal microscopy.

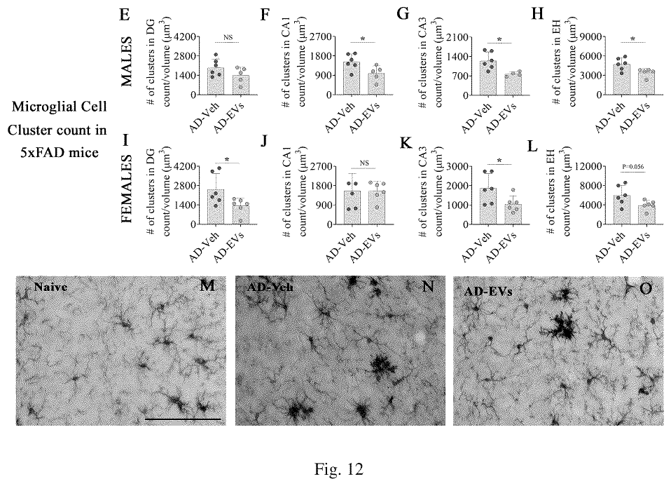

Compared to naïve mice (FIG. 12M), microglial clusters in AD mice imply areas of intense neuroinflammatory changes (FIG. 12N). Intranasal hiPSC-NSC-EV treatment reduced the number of microglial clusters in AD mice (FIG. 12O). Compared to AD mice receiving the vehicle, male AD mice receiving hiPSC-NSC-EVs displayed reduced clusters of microglia in the CA1 and CA3 subfields and when the hippocampus was taken in its entirety (p<0.05; FIGS. 12F-H). Female AD mice receiving hiPSC-NSC-EVs displayed reduced clusters of microglia in the dentate gyrus (DG) and the CA3 subfield (p<0.05; FIGS. 12 I, K).

AD mice receiving the vehicle displayed numerous microglial clusters in different hippocampus regions. However, AD mice receiving hiPSC-NSC-EVs displayed fewer microglial clusters, implying that areas of intense neuroinflammatory changes were reduced in the AD brain following intranasal hiPSC-NSC-EV treatment.

### Example 14—Expression of Disease-Associated Microglia (DAM) Genes

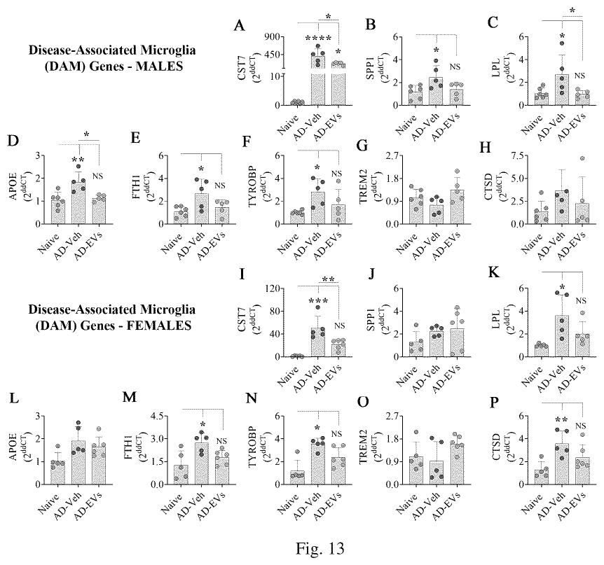

Compared to their counterparts in the naïve group, male AD mice receiving vehicle displayed increased expression of cst7, spp1, lpl, apoe, fth1, tyrobp (p<0.05-0.0001, FIGS. 13A-F) and female AD mice receiving vehicle displayed increased expression of cst7, lpl, fth1, tyrobp, ctsd (p<0.05-0.001, FIGS. 131, K, M, N, P). In contrast, compared to their counterparts in the naïve group, male AD mice receiving hiPSC-NSC-EVs displayed increased expression of only one DAM gene (cst7, FIG. 13A) and female AD mice receiving hiPSC-NSC-EVs did not display increased expression of any of DAM genes measured (p>0.05, FIGS. 13I-P).

Both male and female AD mice receiving vehicle, compared to age-matched male and female naïve mice, displayed increased expression of multiple disease-associated microglia (DAM) genes (FIGS. 13A-P). However, in both male and female AD mice receiving hiPSC-NSC-EVs, the expression of all or the vast majority of DAM genes was normalized to naïve control levels (FIGS. 13A-P). These results imply that modulation of proinflammatory microglia into less inflammatory or non-inflammatory microglia by hiPSC-NSC-EVs observed at 72 hours post-treatment persists for several months.

### Example 15—Expression of Genes Linked to NLRP3 Inflammasome Activation

The expression of genes encoding mediators of NLRP3 inflammasome activation (nlrp3, pycard, casp1) and genes encoding end products of NLRP3 inflammasome activation (il1b, il18) were measured.

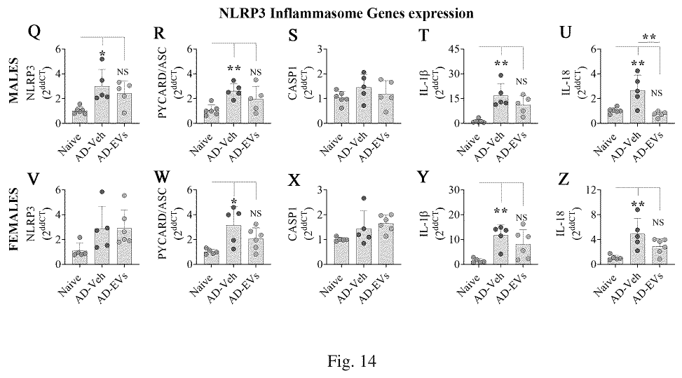

Compared to their counterparts in the naïve group, male AD mice receiving the female AD mice receiving the vehicle displayed increased expression of pycard, il1b, and il18 (p<0.05-0.01, FIGS. 14 W, Y, Z). In contrast, compared to their counterparts in the naïve group, male and female AD mice receiving hiPSC-NSC-EVs did not display increased expression of any of the genes linked to inflammasome activation (p>0.05, FIGS. 14 Q-Z).

Both male and female AD mice receiving vehicle, compared to age-matched male and female naïve mice, displayed increased expression of multiple genes linked to NLRP3 inflammasome activation. However, in both male and female AD mice receiving hiPSC-NSC-EVs, the expression of all inflammasome genes was normalized to naïve control levels. These results imply that hiPSC-NSC-EV treatment in the early phase of AD can significantly restrain the activation of NLRP3 inflammasomes for prolonged periods.

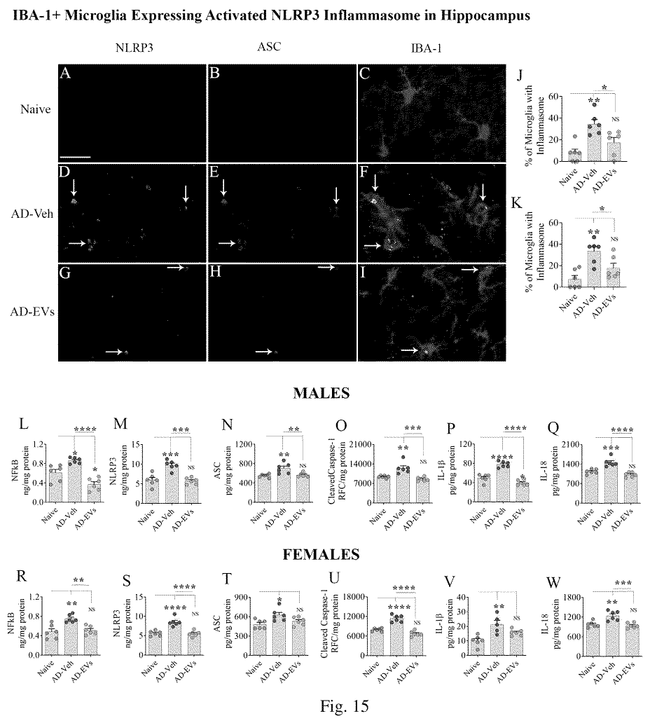

The percentages of microglia displaying NLRP3 inflammasomes (FIGS. 15A-K) and the concentrations of mediators of inflammasomes (NF-kB, NLRP3, ASC, cleaved caspase-1, FIGS. 15L-O, and R-U) and end products of inflammasomes (IL-1b and IL-18, FIGS. 15P-Q, and V-W) were measured.

The percentages of microglia displaying NLRP3 inflammasomes increased in male and female AD mice receiving vehicle, compared to their counterparts in the naïve group (p<0.01, FIGS. 15J-K). However, compared to their counterparts in the naïve group, male and female AD mice receiving hiPSC-NSC-EVs did not display increased percentages of microglia displaying NLRP3 inflammasomes (p>0.05, FIGS. 15Q-Z). Similarly, compared to their counterparts in the naïve group, the concentrations of NF-kB, NF-kB, NLRP3, ASC, cleaved caspase-1 IL-1b, and IL-18 increased in both male and female AD mice receiving vehicle (p<0.05-0.0001, FIGS. 15L-W). In contrast, compared to their counterparts in the naïve group, male and female AD mice receiving hiPSC-NSC-EVs did not display increased concentrations of any of the markers of NLRP3 inflammasomes (p>0.05, FIGS. 15L-W).

Compared to their counterparts in the naïve group, male and female AD mice receiving vehicle displayed significant NLRP3 inflammasome activation. However, in male and female AD mice receiving hiPSC-NSC-EVs, the concentrations of both mediators and end products of NLRP3 inflammasome activation were normalized to naïve control levels or went below naïve control levels. Similar to gene expression results detailed above, these results imply that hiPSC-NSC-EV treatment in the early phase of AD can significantly restrain the activation of NLRP3 inflammasomes for prolonged periods.

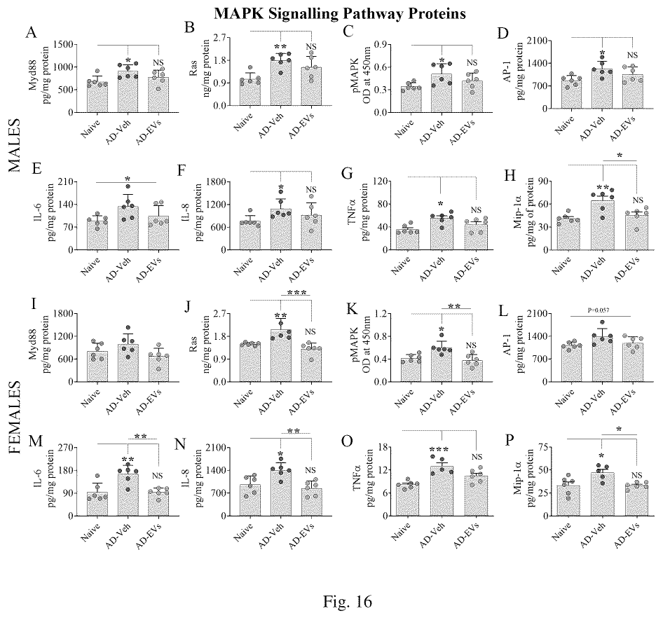

The end products of the inflammasome activation leads to the further downstream activation of p38/MAPK signaling. As such, concentrations of different components of p38/MAPK signaling, including myeloid differentiation primary response 88 (MyD88), a small GTPase rat sarcoma virus (Ras), phospho-p38 MAPK, and activator protein 1 (AP-1) were also measured. Some of the known end products of such signaling, including IL-6, tumor necrosis factor-alpha (TNF-α), and IL-8 and Mip-1a were also measured. The results are shown in FIG. 16.

Compared to their counterparts in the naïve group, the concentrations of MyD88, Ras, phospho-p38/MAPK, AP-1, IL-6, IL-8, TNF-α and Mip-1a increased in male AD mice receiving vehicle (p<0.05-0.01, FIGS. 16A-H) and the concentrations of Ras, phospho-p38/MAPK, IL-6, IL-8, TNF-α and Mip-1a increased in female AD mice receiving vehicle (p<0.05-0.001, FIGS. 16J-K, M-P). In contrast, compared to their counterparts in the naïve group, male and female AD mice receiving hiPSC-NSC-EVs did not display increased concentrations of any of the markers of hyperactivated p38/MAPK signaling (p>0.05, FIGS. 16L-W).

Compared to their counterparts in the naïve group, male and female AD mice receiving the vehicle displayed hyperactivation of p38/MAPK signaling. However, in both male and female AD mice receiving hiPSC-NSC-EVs, the concentrations of all markers of hyperactivation of p38/MAPK signaling were normalized to naïve control levels. These results demonstrate that hiPSC-NSC-EV treatment in the early phase of AD can significantly restrain critical signaling pathways that perpetuate neuroinflammation for prolonged periods.

### Example 16—Investigation of Long-Term Changes in Reactive Astrocytes in 5XFAD Mice after Intranasal Administration of hiPSC-NSC-EVs

The extent of activation of astrocytes through GFAP immunohistochemistry was determined by measuring the area fraction of GFAP+ structures in the hippocampus.

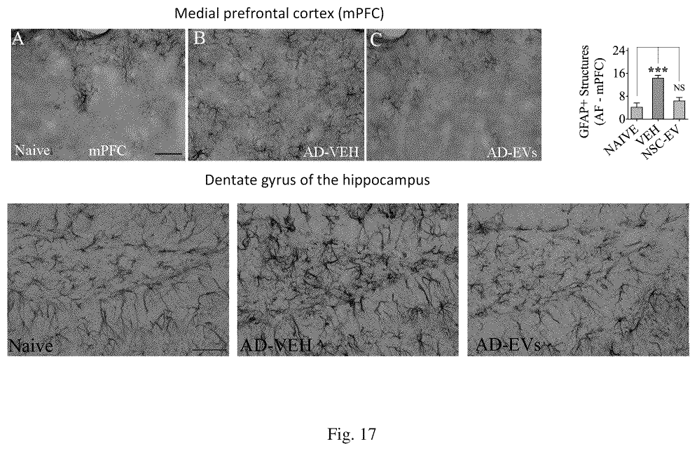

Astrocyte hypertrophy and hyperplasia is the other feature of neuroinflammation in AD. Our results using GFAP immunohistochemistry showed increased astrocyte hypertrophy in the medial prefrontal cortex (mPFC, FIG. 17, upper panel) and the hippocampus (FIG. 17, lower panel) of AD mice receiving vehicle (FIG. 17, middle photograph in both upper and lower panels), compared to naïve mice (photographs on the left in both upper and lower panels of FIG. 17). However, astrocyte hypertrophy was reduced in AD mice receiving hiPSC-NSC-EVs (photographs on the right in both upper and lower panels of FIG. 17). Quantifying the area fraction (AF) of astrocytic elements in the mPFC confirmed these findings (see the bar chart in the upper panel of FIG. 17).

AD mice receiving vehicle, compared to naïve mice, displayed significant astrocyte hypertrophy and hyperplasia in the media prefrontal cortex and the hippocampus. However, AD mice receiving hiPSC-NSC-EVs displayed reduced astrocyte hypertrophy and hyperplasia. These results demonstrate that hiPSC-NSC-EV treatment in the early phase of AD can significantly restrain astrocyte-related changes.

### Example 17—Investigation of Long-Term Changes in Aβ42 and Phosphorylated-Tau Proteins in 5XFAD Mice after Intranasal Administration of hiPSC-NSC-EVs

The extent of amyloid plaques in the brain was measured through Aβ42 immunohistochemistry and imaging. Soluble Aβ42 levels and phosphorylated tau (another pathological hallmark of Alzheimer's disease) in the hippocampus were measured using ELISA.

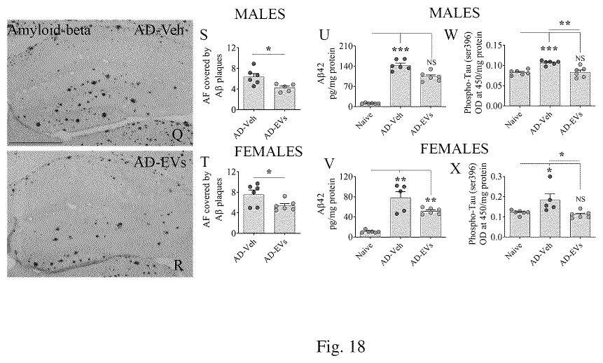

5XFAD mice are known to exhibit significant amounts of amyloid plaques in the hippocampus at 5 months of age. ImageJ analysis of the amyloid plaques showed reduced area fraction (AF) of amyloid plaques in male and female AD mice receiving hiPSC-NSC-EVs, compared to male and female AD mice receiving vehicle (p<0.05, FIGS. 18Q-T). The concentration of soluble A-beta 42 in the hippocampus was considerably increased in male and female AD mice receiving vehicle, compared to the naïve group (p<0.01-0.001, FIG. 18U-V). In male and female AD mice receiving hiPSC-NSC-EVs, A-beta 42 concentrations were reduced compared to their counterparts in the AD-Veh group, but the reductions were not significant statistically (p>0.05;

FIG. 18U-V). Phosphorylation tau (p-tau) showed increased concentration in male and female AD mice receiving vehicle, compared to the naïve group (p<0.05-0.001, FIGS. 18W-X). Remarkably, p-tau levels in male and female AD mice receiving hiPSC-NSC-EVs were normalized to naïve control levels (p>0.05) and significantly reduced in comparison to male and female AD mice receiving vehicle (p<0.05-001, FIGS. 18W-X).

Male and female AD mice receiving hiPSC-NSC-EVs displayed reduced amounts of amyloid plaques compared to male and female AD mice receiving the vehicle. Moreover, compared to their counterparts in the naïve group, male and female AD mice receiving vehicle displayed increased concentration of p-tau in the hippocampus. However, p-tau levels in AD mice receiving hiPSC-NSC-EVs were normalized to naïve control levels. These results demonstrate that hiPSC-NSC-EV treatment in the early phase of AD can reduce amyloid plaque deposition and p-tau levels.

### Example 18—Investigation of Hippocampal Neurogenesis in 5XFAD Mice after Intranasal Administration of hiPSC-NSC-EVs

The status of hippocampal neurogenesis through doublecortin (DCX) immunostaining and stereological quantification of DCX+ newly born neurons in the subgranular zone-granule cell layer (SGZ-GCL) of the hippocampus.

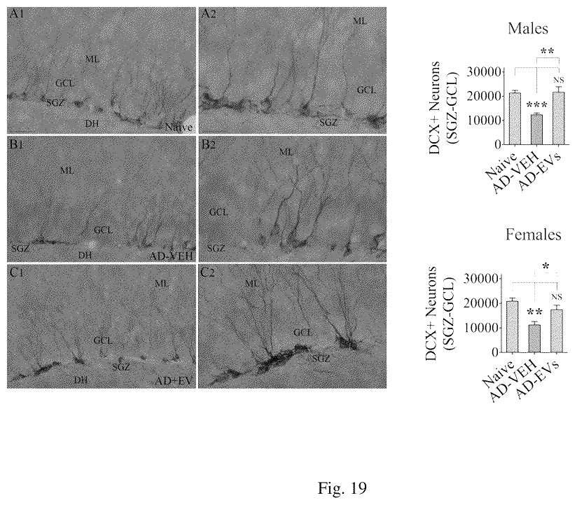

Decreased hippocampal neurogenesis is another pathological hallmark of AD. DCX immunohistochemistry results showed decreased hippocampal neurogenesis in AD mice receiving vehicle (FIGS. 19 B1-B2) compared to naïve mice (FIGS. 19A 1-A2). However, in AD mice receiving hiPSC-NSC-EVs (FIGS. 19 C1-C2), neurogenesis did not decline. Stereological quantification revealed a significant decline in the number of DCX+ newly born neurons in the SGZ-GCL of male and female AD mice compared to their counterparts in the naïve group (p<0.01-0.001, bar charts on the right side). However, in male and female AD mice receiving hiPSC-NSC-EVs, neurogenesis was maintained at naïve control levels (p>0.05, bar charts on the right side).

Male and female AD mice receiving vehicle, compared to their counterparts in the naïve group, displayed a significant decline in hippocampal neurogenesis. However, male and female AD mice receiving hiPSC-NSC-EVs maintained normal levels of hippocampal neurogenesis. These results demonstrate that hiPSC-NSC-EV treatment in the early phase of AD can significantly restrain the decline in hippocampal neurogenesis.

### Example 19— Treatment of Patient with Alzheimer's Disease

hiPSCs are obtained from the patient with Alzheimer's disease. NSCs are generated therefrom and EVs isolated from the media used to culture the NSCs. The patient is treated with EVs released by hiPSC-derived NSCs. The patient receives a single intravenous injection of about 6.5×10¹¹ vesicles per kg of body weight of the patient.

### Example 20— Treatment of Patient with Alzheimer's Disease

hiPSCs are obtained from the patient with Alzheimer's disease. NSCs are generated therefrom and EVs isolated from the media used to culture the NSCs. The patient is treated with EVs released by hiPSC-derived NSCs. The patient receives an intranasal administration of about 6.5×10¹¹—vesicles per kg of body weight of the patient.

1. A method for treating a neurological disorder, comprising administering a therapeutically effective amount of extracellular vesicles (EVs) derived from human induced pluripotent stem cell derived neural stem cells (hiPSC-NSCs) to a subject in need thereof.

2. The method of claim 1, wherein the subject is a human.

3. The method of claim 1, wherein the neurological disorder is Alzheimer's disease, Parkinson's disease, Huntington's disease, amyotrophic later sclerosis (ALS), multiple sclerosis, dementia, traumatic brain injury, or stroke.

4. The method of claim 1, wherein the neurological disorder is Alzheimer's disease.

5. The method of claim 1, wherein the EVs are administered by injection, intranasal delivery, convection-enhanced delivery, oral administration, or infusion.

6. The method of claim 1, wherein the EVs are administered by intranasal delivery.

7. The method of claim 1, wherein the EVs are administered in a dose range of about 1×10⁹ to about 1×10¹⁴ vesicles per kg of body weight of the subject.

8. The method of claim 1, wherein the EVs are administered in a dose of about 6.5×10¹¹ vesicles per kg of body weight of the subject.

9. The method of claim 1, wherein the administration is repeated over a period of time to provide sustained therapeutic benefit to the subject.

10. The method of claim 1, wherein the EVs are modified to increase their stability in vivo.

11. The method of claim 1, wherein the EVs are modified to increase their targeting to the brain.

12. The method of claim 11, wherein the EVs are modified by attaching a targeting moiety.

13. The method of claim 1, wherein the EVs are modified to increase their therapeutic efficacy.

14. The method of claim 1, wherein the EVs comprise a therapeutic agent.

15. The method of claim 1, wherein the EVs are administered in combination with another therapeutic compound.

16. The method of claim 15, wherein the other therapeutic compound is a cholinesterase inhibitor, a memantine, an acetylcholine agonist, or a gamma-secretase inhibitor.

17. A composition comprising extracellular vesicles (EVs) derived from human induced pluripotent stem cell derived neural stem cells (hiPSC-NSCs).

18. The composition of claim 17, further comprising a carrier.

19. The composition of claim 18, wherein the carrier comprises phosphate buffered saline.

20. A kit for use in treating a neurological disorder comprising: a plurality of EVs derived from hiPSC-NSCs; and instructions for administering the EVs to a subject.
